(19) United States
(12) Patent Application Publication
(10) Pub. No.: US 2008/0294187 A1
(43) Pub. Date: Nov. 27, 2008

(54) Neurosurgical Balloon Retractor

(75) Inventor: Ali Krisht, Little Rock, AR (US)

Correspondence Address: HARNESS, DICKEY & PIERCE, P.L.C., P.O. BOX 828, BLOOMFIELD HILLS, MI, 48303, US

(73) Assignee: BIOMET MICROFIXATION, LLC, Jacksonville, FL (US)

(21) Appl. No.: 12/101,563

(22) Filed: Apr. 11, 2008

Related U.S. Application Data
(60) Provisional application No. 60/911,743, filed on Apr. 13, 2007.
(60) Provisional application No. 60/923,704, filed on Apr. 16, 2007.

(51) Int. Cl. A61M 29/02

(52) U.S. Cl. 606/192

(57) ABSTRACT

A method of performing surgery can include, identifying a line of resection on tissue. A bladder can be placed adjacent to the line of resection. The bladder can be inflated to form a barrier against tissue along the line of resection.

### CROSS-REFERENCE TO RELATED APPLICATIONS

This application claims the benefit of U.S. Provisional Application No. 60/911,743, filed on Apr. 13, 2007. This application claims the benefit of U.S. Provisional Application No. 60/923,704, filed on Apr. 16, 2007. The disclosures of the above applications are incorporated herein by reference.

### FIELD

The present disclosure relates generally to neurosurgery, and more specifically, to a method and apparatus for providing a protective barrier and/or maintaining an anatomical space.

### BACKGROUND

In some instances, a human brain may be functioning improperly due to undeveloped or otherwise dysfunctional areas such as those causing seizures for example. In other examples, abnormal growths such as tumors and/or aneurysms may be detected in the brain.

In these examples and others, it may be necessary to surgically dissect and/or resect such portions of the brain. During such a procedure, the pressure effect of microinstruments on the surface of the brain may be attenuated by forming a barrier between such microinstruments and brain tissue. During the process of separating a growth (such as a tumor, etc.) from brain tissue, it is common for a neurosurgeon to maintain the space created between the brain and the removed portion.

Conventionally, cottonoids can be placed between the microinstruments and the brain during such a procedure. Cottonoids, which are generally small square or rectangular pieces of absorbent material with a long string attached, can be used in neurosurgical procedures for packing and hemostasis of small tissues. If it adheres to the surface of the brain-, a cottoned may injure the pial surface of the brain when removed. Cottonoids also have a fixed size and shape, which requires that the correct size of cottonoid be estimated correctly for use during the neurosurgical procedure.

### SUMMARY

A method of performing surgery can include, identifying a line of resection on tissue. A bladder can be placed adjacent to the line of resection The bladder can be inflated to form a barrier against tissue along the line of resection.

According to additional features, placing the bladder can include locating the bladder against exposed tissue at the line of resection subsequent to resection. Placing the bladder can include placing a bladder having a non-stick surface against the tissue. Inflating the bladder can include inflating the bladder along at least one geometrical dimension, while maintaining at least another geometrical dimension substantially fixed. According to other features, a plurality of bladders can be provided each having a distinct fixed dimension and one of the bladders can be selected based on a characteristic of the line of resection. The characteristic can include a depth of resection.

According to some examples, inflating the bladder can include delivery fluid through a cannulated member extending between the bladder and a fluid source. Inflating the bladder can include depressing a plunger disposed at the fluid source. Inflating the bladder can include inflating the bladder hydraulically. Inflating the bladder can include inflating the bladder pneumatically. Inflating the bladder can include maintaining an anatomical space between first tissue on one side of the line of resection and second tissue on another side of the line of resection.

A method of performing surgery according to other features can include, resecting a first tissue portion from a second tissue portion along a line of resection with a cutting instrument. A bladder can be placed adjacent to exposed tissue of the second tissue portion. The bladder can be inflated to a first size, thereby forming a protective barrier between the exposed tissue and the resected first tissue.

According to additional features, the first tissue portion can be further resected from the second tissue portion along the line of resection with the cutting instrument. The bladder can be inflated to a second size between the exposed tissue and the resected first tissue, the second size being greater than the first size. The bladder can be located against the exposed tissue formed at the resection line subsequent to resection. Placing the bladder can include placing a bladder having a non-stick surface against the exposed tissue. Inflating the bladder can include inflating the bladder along at least one geometrical dimension while maintaining at least another geometrical dimension substantially fixed. In one example, inflating the bladder can include delivering fluid from a cannulated member extending between the bladder and a fluid delivery portion. Inflating the bladder can include depressing a plunger disposed at the fluid delivery portion. Inflating the bladder can include inflating the bladder hydraulically.

An apparatus for assisting a surgical procedure can include an inflatable bladder, a fluid delivery portion, and a cannulated member fluidly connected between the inflatable bladder and the delivery portion. The inflatable bladder can be adapted to inflate along at least one geometrical dimension while maintaining at least another geometrical dimension substantially fixed.

According to additional features, the fluid delivery portion can include a plunger slidably disposed within a syringe. The inflatable bladder can be formed of a non-stick material. The inflatable bladder can be formed of silicone. The cannulated member can be flexible.

### BRIEF DESCRIPTION OF THE DRAWINGS

The present teachings will become more fully understood from the detailed description and the accompanying drawings, wherein:

FIG. 1A is a perspective view of an apparatus for maintaining an anatomical space adjacent brain tissue during a neurosurgical procedure, the apparatus generally including an inflatable balloon or bladder (shown uninflated), a delivery portion and a cannulated member extending therebetween;

FIG. 1B is a perspective view of the apparatus of FIG. 1A shown with a plunger of the delivery portion partially depressed and the bladder partially inflated to a first size;

FIG. 1C is a perspective view of the apparatus of FIG. 1B shown with a plunger of the delivery portion depressed further and the bladder inflated further to a second size;

FIG. 2 is a side view of a patient's head with the brain shown in phantom and having a portion of unhealthy tissue identified generally on the frontal lobe for exemplary purposes;

FIG. 3 is an enlarged perspective view of a surgical site shown with a portion of the cranium removed and the bladder initially placed adjacent to the unhealthy tissue;

FIG. 4 is an enlarged perspective view of the surgical site of FIG. 3 shown with the unhealthy tissue partially resected with a surgical instrument and the bladder partially inflated generally between the portion of resected unhealthy tissue and the newly exposed healthy tissue;

FIG. 5 is an enlarged perspective view of the surgical site of FIG. 4 shown with the unhealthy tissue resected further with the surgical instrument and the bladder inflated further to occupy an anatomical space between the resected unhealthy tissue and the newly exposed healthy tissue;

FIG. 6 is a perspective view of an apparatus according to additional features for maintaining an anatomical space adjacent brain tissue during a neurosurgical procedure, the apparatus generally including an inflatable balloon or bladder (shown uninflated or partially inflated in solid line and inflated further in phantom line), a delivery portion and a cannulated member extending therebetween;

FIG. 7 is a perspective view of a surgical site shown with the bladder of FIG. 6 initially placed at a desired location;

FIG. 8 is a perspective view of the surgical site of FIG. 7 with another bladder initially placed at a desired location;

FIG. 9 is a perspective view of the surgical site of FIG. 8 shown with the pair of bladders inflated to a desired size to create an anatomical space therebetween; and

FIG. 10 is a perspective view of a kit having the apparatus of FIG. 6 according to one example.

### DETAILED DESCRIPTION OF VARIOUS EMBODIMENTS

The following description of various embodiments is merely exemplary in nature and is in no way intended to limit the teachings, its application, or uses. By way of example, the following description is directed toward a neurosurgical tumor resection of the frontal lobe. It is appreciated however that the same may be used during resection of unhealthy tissue located elsewhere in the brain. It is further appreciated that the following may be used for other surgeries such as orthopedic procedures, spinal procedures, cardiac, procedures, other neurological procedures or any other surgical or medical procedure. As used herein, the term unhealthy tissue is used generally to denote tissue to be removed (i.e., a tumor, a growth, or otherwise undesirable tissue) and the term healthy tissue is used generally to denote tissue to remain in situ.

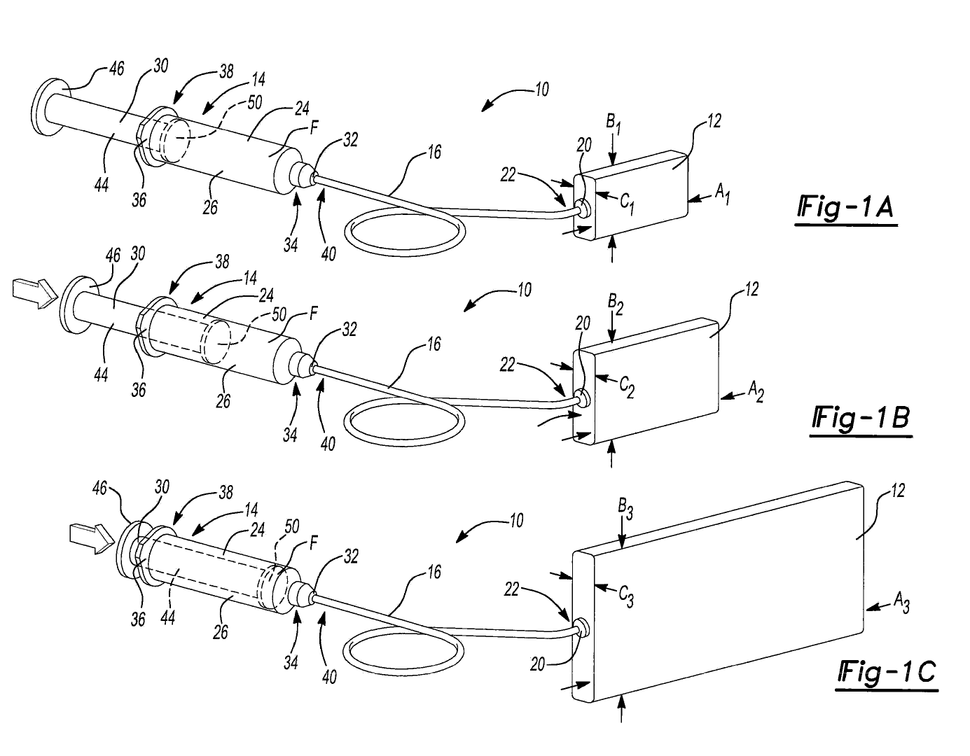

With initial reference to FIGS. 1A-1C, an apparatus constructed in accordance to one example of the present teachings is shown and generally identified at reference 10. The apparatus 10 can generally include an inflatable portion or bladder 12, a delivery portion 14 and a cannulated member 16 fluidly connected therebetween. The exemplary bladder 12 shown is generally in the form of a rectangle having a first, second and third dimension A, B and C respectively. It is appreciated however that the bladder may take the form of other geometrical configurations such as, but not limited to, cylindrical. In one example, the bladder 12 can be adapted to expand along one or more dimensions (such as along dimensions A and B in the example shown) upon the introduction of fluid. In addition, the bladder 12 can also be adapted to be fixed along one or more dimensions during expansion (such as along dimension C in the example shown). In one example, the bladder 12 can be formed of a non-stick, biocompatible material such as silicone. Other materials may be used such as natural rubber, latex, and latex-free rubber for example. The bladder 12 can define an inlet 20 fluidly connected to a first end 22 of the cannulated member 16.

The delivery portion 14 can generally include a syringe 24 having an internal chamber 26 and a plunger 30. The syringe 24 can define an outlet 32 at a first end 34 and a handle 36 at an opposite end 38. The outlet 32 can be fluidly connected to a second end 40 of the cannulated member 16. The plunger 30 can include a guide shaft 44 disposed between a plunger button 46 and a piston 50. In an assembled position (as shown), an outer dimension of the piston 50 can form a fluid tight seal with an inner dimension of the internal chamber 26 of the syringe 24. As can be appreciated, depression of the plunger button 46 toward the syringe outlet 32 can cause the piston 50 to urge fluid F from the internal chamber 26 and into the cannulated member 16. From the cannulated member 16, the fluid F can be urged into the inlet 20 of the bladder 12 causing the bladder 12 to inflate.

Any suitable fluid can be used. In one example, liquid such as water may be used to inflate the bladder 12 hydraulically. Other liquids are contemplated. In another example, air may be used to inflate the bladder 12 pneumatically.

With specific reference now to FIGS. 1A-1C, an exemplary sequence of inflation will be described. In FIG. 1A, the bladder 12 is shown in an original generally uninflated state. Turning to FIG. 1B, the plunger button 46 is partially depressed causing an amount of fluid F to be directed into the bladder 12. As illustrated, the dimensions A₁ and B₁ have increased to dimensions A₂ and B₂, respectively, due to inflation whereas the dimension C₂ remains substantially unchanged or fixed from dimension C₁. Moving to FIG. 1C, the plunger button 46 is depressed further causing additional fluid to be directed into the bladder 12. As illustrated, the dimensions A₂ and B₂ have increased to dimensions A₃ and B₃, respectively, due to inflation whereas the dimension C₃ remains substantially unchanged or fixed from dimension C₂. In sum, the exemplary bladder 12 can be adapted to inflate under the following geometrical attributes: A₁<A₂<A₃; B₁<B₂<B₃; and C₁=C₂=C₃. It is appreciated however, that the bladder 12 may be configured to inflate differently. For example, only one of the dimensions may be adapted to expand or all of the dimensions may be adapted to expand. Likewise, bladders having other configurations, such as cylindrical may be adapted to expand along an elongated axis while remaining substantially fixed about a circular cross-section. In another example, a substantially spherical bladder may be provided that expands generally uniformly in all directions. Other geometrical shapes having distinct expansion properties may be provided.

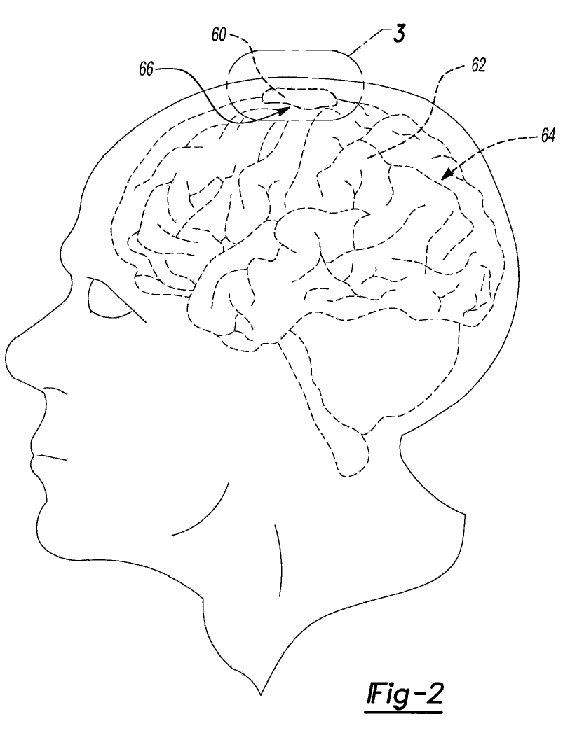

With reference now to FIG. 2, an exemplary neurosurgical procedure will be described. At the outset, a neurosurgeon can identify the tissue which is to be resected. While such identification is generally outside the scope of this disclosure, many neurosurgical procedures utilize patient images obtained prior to or during a medical procedure to guide a physician performing the procedure. Such procedures can be referred to as computer assisted procedures. Some examples of imaging technology can produce highly-detailed, two, three, and four dimensional images, such as computed tomography (CT), magnetic resonance imaging (MRI), fluoroscopic imaging (such as with an O-arm device), positron emission tomography (PET), and ultrasound imaging (US). Nonetheless, in the example shown, the unhealthy tissue is generally identified at reference 60 while the healthy tissue is generally identified at reference 62. The brain as a whole is identified at reference 64.

Once the unhealthy and healthy tissue 60 and 62, respectively, have been distinguished, a neurosurgeon can identify a location where the unhealthy tissue 60 can be resected from the healthy tissue 62. For discussion purposes, this location will be referred generally to a line of resection 66. Those skilled in the art will appreciate that the term “line” is used generally because the act of resection may not necessarily occur along a line, but rather may occur along any linear or non-linear path or area. Likewise, the act of resection may be defined along a thickness of tissue wherein the resection itself may partially or completely involve vaporization of unhealthy tissue such as when using a surgical laser. In such an example, the line of resection may comprise completely, or partially an area of vaporization.

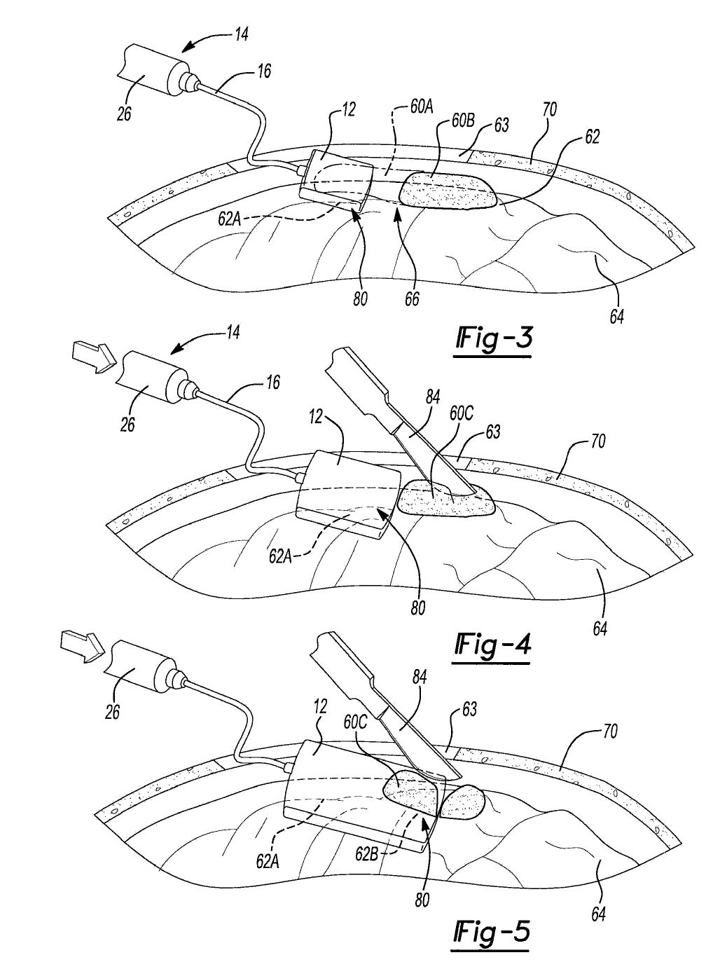

Turning now to FIGS. 3-5, an exemplary method of using the apparatus 10 will be described. It will be appreciated that the scale of the inflatable bladder 12 (and cutting instrument 84) may be enlarged in relation to the brain 64 simply to show detail. When performing surgery on the brain 64, it is often necessary to perform a craniotomy to provide access to portions of the brain 64. A craniotomy is a surgical procedure in which a portion of a cranium 70 is removed to permit access to the brain 64. To perform the craniotomy, one or more holes (not shown) can be initially drilled through the cranium 70. These holes, known as “burr holes,” may be located, for example, at the corners of a triangular region of bone that is to be temporarily removed. A saw, i.e., a craniotome, can then used to cut the cranium 70 along the line of separation connecting the adjoining burr holes. The resulting bone cover or bone plug (not shown) can be subsequently lifted from the underlying dura matter to define a passage 63 and expose the brain 64. The bone cover may either be completely removed from the cranium 70, or folded back in a flap along an uncut edge of the flap.

Once access to the brain 64 has been created, resection of the unhealthy tissue 60 can now be performed. As shown in FIG. 3, a portion of unhealthy tissue 60 (identified in phantom at reference 60A) has been resected. A reminder of the unhealthy tissue 60 still remains and is identified generally at reference 60B). According to one example, a plurality of inflatable bladders 12 can be provided, each having distinct geometrical shapes and expansion characteristics. In this way, a neurosurgeon can select an appropriate bladder 12 given the relative size of the unhealthy tissue 60 and bordering healthy tissue 62.

As will become appreciated from the following discussion, the bladder 12 can be used to form a protective barrier and/or maintain an anatomical space adjacent healthy tissue 62 during a tissue resection procedure. In addition, the bladder 12 can be used to gently retract, elevate and/or hold neutral tissues during a surgical procedure. In this way, the bladder can improve access to pathologies with minimal disruption of the brain tissue. Explained differently, the apparatus 10 can be used to enhance microsurgical techniques, help provide atraumatic exposure and minimize retraction trauma by minimizing injury to surrounding neural structures. It should further be noted that the bladder 12 may be used to provide a protective barrier from any type of instrument, delivery system or other objects used by a surgeon near the brain 64 during a procedure, including but not limited to: biopsy needles, surgical lasers, ultrasonic aspirators, arthroscopic systems, operating microscopes, deep brain stimulation (DBS) probes etc.

At this point, the selected bladder 12 can be positioned adjacent to newly exposed healthy brain tissue identified at reference 62A. The bladder 12 may be uninflated or partially inflated to occupy an anatomical space 80 adjacent to the newly exposed healthy tissue 62A. While not necessary, the bladder 12 can rest against the newly exposed healthy tissue 62A. In addition to or alternatively, the bladder 12 can be used to occupy an anatomical space 80 adjacent to undisturbed healthy tissue 62 (i.e., brain tissue not along the line of resection).

With reference to FIG. 4, the bladder 12 is shown further inflated to form a protective barrier adjacent to the healthy tissue 62A. Next, additional unhealthy tissue 60C can be resected by the cutting instrument 84. It is appreciated that while the cutting instrument 84 shown is generally in the form of a scalpel, additional or other instruments, such as those described above may be used to resect the unhealthy tissue 60.

Turning to FIG. 5, the bladder 12 can be further inflated to form a protective barrier adjacent to additional newly exposed healthy tissue 62B. In addition, as shown in FIG. 5, the bladder 12 can be used to maintain an anatomical space between the additional newly exposed healthy tissue 62B and the portion of unhealthy tissue 60C being resected. As can be appreciated, by maintaining such an anatomical space, access to the desired line of resection 66 can be facilitated or enhanced by inflation of the bladder 12. In one exemplary method of use, a neurosurgeon can alternately and sequentially cut a portion of the unhealthy tissue 60 with the instrument 84 and, inflate the bladder 12 further. The sequence can continue until the unhealthy tissue 60 has been completely resected. Once all of the unhealthy tissue 60 has been resected, the bladder 12 can be removed from the surgical site. As explained above, the bladder 12 can be formed of non-stick material. In this way, the bladder 12 can be successfully separated from the healthy tissue 62 with no or minimal abrasion caused to the healthy tissue 62. The bladder 12 can be deflated prior to or subsequent to removal from the surgical site. In one example, the bladder 12 can be deflated by retracting the plunger button 46 thereby siphoning some or all of the fluid F back into the internal chamber 26 of the syringe 24.

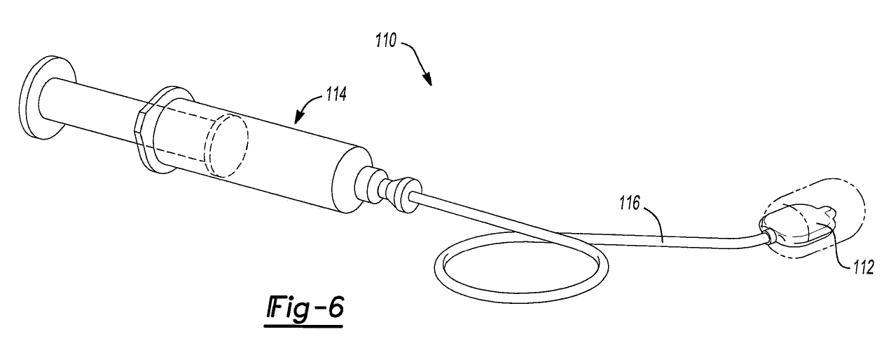

Turning now to FIGS. 6-10, an apparatus 110 and method according to additional features will be described. For simplicity, like reference numerals having a 100 prefix are used to denote like features relative to the apparatus 10.

With initial reference to FIG. 6, the apparatus 110 can generally include an inflatable portion or bladder 112, a delivery portion 114 and a cannulated member 116 fluidly connected therebetween. The apparatus 110 may be constructed substantially similar to the apparatus 10. According to this example, the bladder 112 is generally cylindrical and adapted to expand generally along its elongated axis. As illustrated in FIG. 6, the bladder 112 may expand from a position as shown in solid line to a position as shown in phantom. In one example, the cylindrical bladder 112 can be substantially fixed about a circular cross-section.

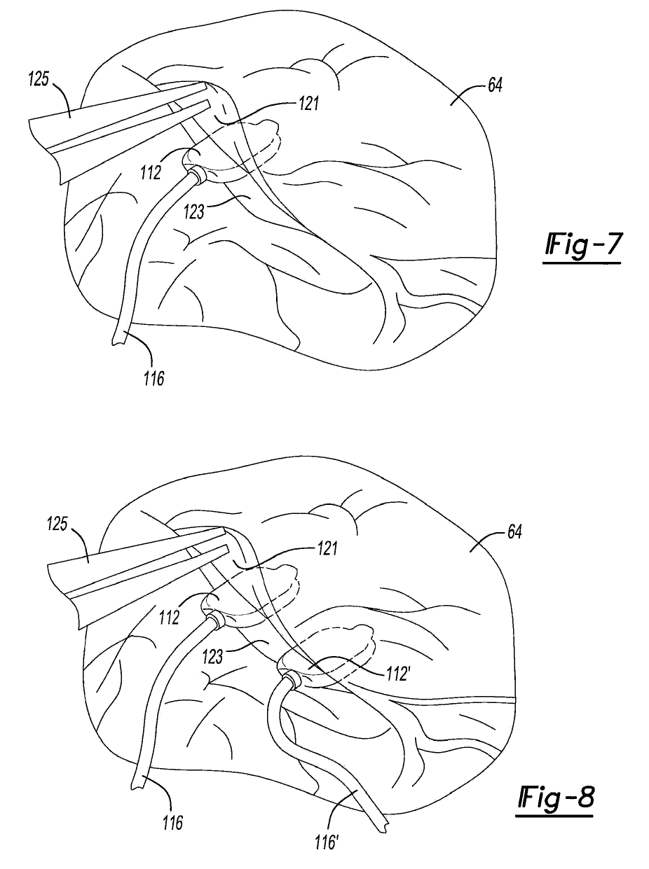

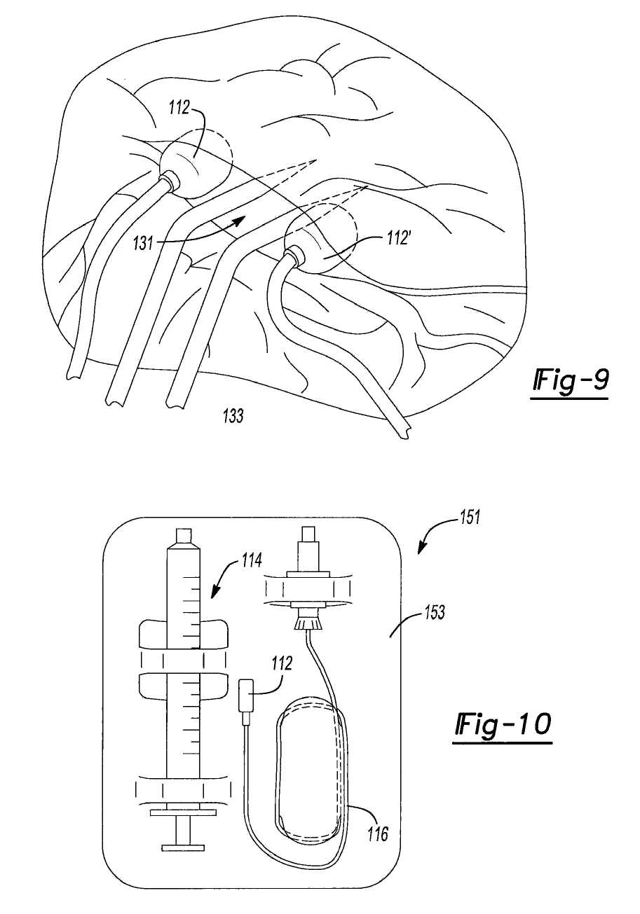

Turning now to FIG. 7, the bladder 112 is shown initially located in the brain 64 between a first tissue portion 121 and a second tissue portion 123. A tool 125 may be used to gain access to a desired location. As shown in FIG. 8, another bladder 112′ is shown initially located in the brain 64 between the first tissue portion 121 and the second tissue portion 123. Again, it will be appreciated that the scale of the inflatable bladders 112 and 112′ (and tool 125) may be enlarged in relation to the brain 64 simply to show detail. The bladder 112′ may also include a cannulated member 116′. While not necessary, the cannulated members 116 and 116′ may be fluidly connected to a common delivery device 114. As such, according to one example, both of the bladders 112 and 112′ may be inflated by a common delivery portion 114. As illustrated in FIG. 9, the bladders 112 and 112′ have both been inflated to a desired size therefore creating an anatomical space 131. Once the anatomical space 131 has been created, an instrument 133 may gain access between the anatomical space 131 to a desired location.

With reference to FIG. 10, a kit 151 may be provided having a carrying case 153. The carrying case can securely store the various items such as the delivery portion 114, bladder 112 and cannulated member.

Further areas of applicability of the present teachings will become apparent from the detailed description provided above. It should be understood that the detailed description and specific examples, while indicating various embodiments are intended for purposes of illustration only and are not intended to limit the scope of the teachings.

What is claimed is:

1. A method of performing surgery, comprising:
identifying a line of resection on tissue;
placing a bladder adjacent to the line of resection; and
inflating the bladder to form a barrier against tissue along the line of resection.

2. The method of claim 1 wherein placing the bladder includes locating the bladder against exposed tissue along the line of resection subsequent to resection.

3. The method of claim 1 wherein placing the bladder includes placing a bladder having a non-stick surface against the tissue.

4. The method of claim 1 wherein inflating the bladder includes inflating the bladder along at least one geometrical dimension while maintaining at least another geometrical dimension substantially fixed.

5. The method of claim 4, further comprising:
providing a plurality of bladders each having a distinct fixed dimension; and
selecting one of the plurality of bladders based on a characteristic of the line of resection.

6. The method of claim 5 wherein the characteristic includes a depth of resection.

7. The method of claim 1 wherein inflating the bladder includes delivering fluid through a cannulated member extending between the bladder and a fluid source.

8. The method of claim 7 wherein inflating the bladder includes depressing a plunger disposed at the fluid source.

9. The method of claim 8 wherein inflating the bladder includes inflating the bladder hydraulically.

10. The method of claim 8 wherein inflating the bladder includes inflating the bladder pneumatically.

11. The method of claim 1 wherein inflating the bladder includes maintaining an anatomical space between first tissue on one side of the line of resection and second tissue on another side of the line of resection.

12. A method of performing surgery, comprising:
resecting a first tissue portion from a second tissue portion along a line of resection with a cutting instrument;
placing a bladder adjacent to exposed tissue of the second tissue portion; and
inflating the bladder to a first size thereby forming a protective barrier between the exposed tissue and the resected first tissue.

13. The method of claim 12, further comprising:
resecting further the first tissue portion from the second tissue portion along the line of resection with the cutting instrument; and
inflating the bladder to a second size between the exposed tissue and the resected first tissue, the second size being greater than the first size.

14. The method of claim 12 wherein placing the bladder includes locating the bladder against the exposed tissue formed at the resection line subsequent to resection.

15. The method of claim 12 wherein placing the bladder includes placing a bladder having a non-stick surface against the exposed tissue.

16. The method of claim 12 wherein inflating the bladder includes inflating the bladder along at least one geometrical dimension while maintaining at least another geometrical dimension substantially fixed.

17. The method of claim 12 wherein inflating the bladder includes delivering fluid through a cannulated member extending between the bladder and a fluid delivery portion.

18. The method of claim 16 wherein inflating the bladder includes depressing a plunger disposed at the fluid delivery portion.

19. The method of claim 16 wherein inflating the bladder includes inflating the bladder hydraulically.

20. An apparatus for assisting a surgical procedure comprising:
an inflatable bladder;
a fluid delivery portion;
a cannulated member fluidly connected between the inflatable bladder and the fluid delivery portion;
wherein the inflatable bladder is adapted to inflate along at least one geometrical dimension while maintaining at least another geometrical dimension substantially fixed.

21. The apparatus of claim 20 wherein the fluid delivery portion includes a plunger slidably disposed within a syringe.

22. The apparatus of claim 21 wherein the inflatable bladder is formed of non-stick material.

23. The apparatus of claim 22 wherein the inflatable bladder is formed of silicone.

24. The apparatus of claim 20 wherein the cannulated member is flexible.
